(19) United States
(12) Plant Patent Application Publication
(10) Pub. No.: US 2014/0123358 P1
(43) Pub. Date: May 1, 2014

(54) Lobelia plant named 'KLELE12473'

(50) ;

(71) Applicant: KLEMM+SOHN GMBH & CO. KG, Stuttgart (DE)

(72) Inventors: Nils Klemm, Stuttgart (DE); Guido Von Tubeuf, Stuttgart (DE)

(73) Assignee: KLEMM+SOHN GMBH & CO. KG, Stuttgart (DE)

(21) Appl. No.: 13/694,078

(22) Filed: Oct. 26, 2012

(51) Int. Cl. A01H 5/00

(52) U.S. Cl. PLT/451

(57) ABSTRACT

Anew and distinct lobelia plant named ‘KLELE12473’ particularly distinguished by white petals with blue corolla tubes and early flowering time, is disclosed.

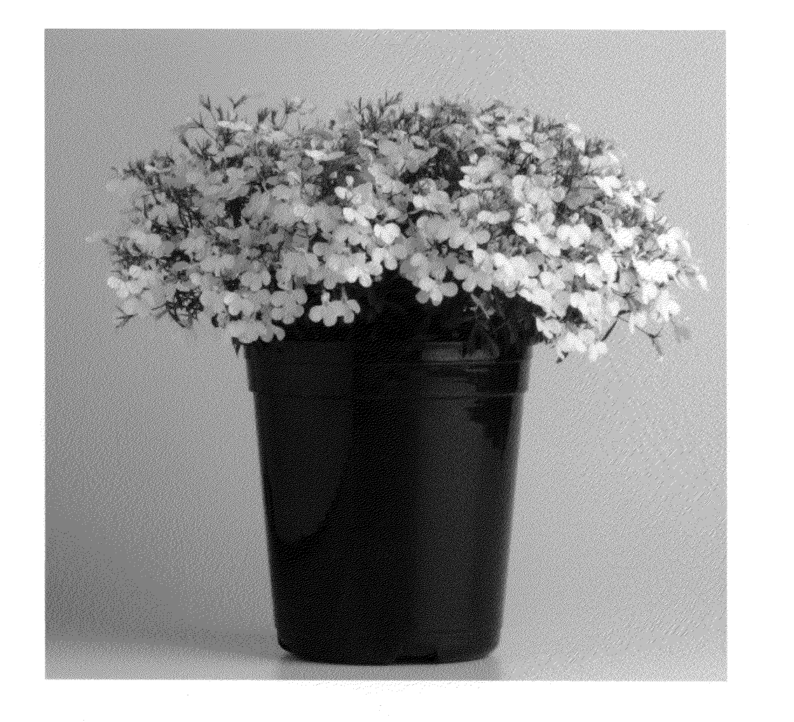

### GENUS AND SPECIES

Lobelia erinus

### VARIETY DENOMINATION

‘KLELE12473’

### BACKGROUND OF THE NEW PLANT

The present invention comprises a new and distinct variety of lobelia, botanically known as Lobelia erinus, and hereinafter referred to by the variety name ‘KLELE12473’. The new variety resulted from a controlled cross conducted in June 2006 in Stuttgart, Germany, between female parent lobelia plant named ‘LE 05 0429’, (unpatented) and an unnamed male parent lobelia plant. A single plant selection was subsequently chosen for further evaluation and asexual propagation.

The new variety was first propagated via vegetative cuttings and in vitro propagation in May of 2007 in Stuttgart, Germany, and has been asexually reproduced repeatedly by vegetative cuttings for 15 generations. ‘KLELE12473’ has been found to retain its distinctive characteristics through successive asexual propagations via vegetative cuttings.

Plant Breeder's Rights for this variety have not been applied for. ‘KLELE12473’ has not been made publicly available more than one year prior to filing of this application.

### SUMMARY OF THE INVENTION

The following are the most outstanding and distinguishing characteristics of this new variety when grown under normal horticultural practices in a glass greenhouse in Stuttgart, Germany.

1. White petals with blue corolla tubes; and

2. Early flowering time.

### DESCRIPTION OF PHOTOGRAPH

This new lobelia plant is illustrated by the accompanying photograph which shows overall plant habit including blooms, buds, and foliage of the plant in full color. The colors shown are as true as can be reasonably obtained by conventional photographic procedures. The photograph was taken in spring of 2012 on a 3-month old lobelia plant grown in a 10 centimeter pot in a glass greenhouse in Stuttgart, Germany under normal horticultural practices.

### DESCRIPTION OF THE NEW VARIETY

The following detailed descriptions set forth the distinctive characteristics of ‘KLELE12473’. The data which define these characteristics were collected from asexual reproductions carried out in Stuttgart, Germany. The plant history was taken in the spring of 2012 on 3-month old plants grown in 10 centimeter pots in a glass greenhouse with a pinch date at week eight. Color readings were taken under natural light. Color references are primarily to the R.H.S. Colour Chart of The Royal Horticultural Society of London (R.H.S.), Fifth Edition (2007).

### DETAILED BOTANICAL DESCRIPTION OF THE NEW PLANT

Classification:
   Botanical.—Lobelia erinus.
   Common name.—Lobelia.
   Designation.—‘KLELE12473’.
Parentage:
   Female parent.—Lobelia plant named ‘LE 05 0429’ (unpatented).
   Male parent.—Unnamed.
Growth:
   Growth and branching habit.—Half-hanging.
   Form.—Rounded to flat.
   Height (measured from the top of the soil, including any flowers).—10.0 cm to 12.0 cm.
   Width (horizontal plant diameter, including any flowers).—10.0 cm to 12.0 cm.
   Time to produce a finished flowering plant.—8 weeks.
   Outdoor plant performance.—Very good.
   Time to initiate roots.—3 weeks.
   Root description.—Fine, white roots, freely branching and dense.
Leaves:
   Arrangement.—Alternate.
   Length.—2.0 cm.
   Width.—0.3 cm.
   Shape.—Elliptical to lanceolate.
   Margin.—Serrate.
   Apex.—Obtuse.
   Base.—Acute.
   Texture (both upper and lower surfaces).—Smooth.
   Color (immature and mature leaves).—Upper surface: RHS 137A Lower surface: RHS 137C.
   Venation.—Pinnate.
Stems:
   Length.—10.0 cm.
   Diameter.—0.2 cm.
   Internode length.—1.0 cm to 1.5 cm.
   Color.—RHS 137B.
   Texture.—Smooth.
   Stem anthocyanin.—Absent.
   Peduncle.—Color: RHS 137B Length: 1.0 cm to 3.0 cm Diameter: 0.05 cm Texture: Smooth.
Flower buds:
   Color (when first opening).—Bluish at the apex with yellow to greenish white at the base.
   Shape.—Inverse drop.
   Diameter.—0.25 cm.
   Length.—0.8 cm.
Inflorescence:
   Blooming habit.—Continuous.
   Lastingness of individual blooms on plant.—Approximately 1 week.
   Fragrance.—Absent.
   Inflorescence type.—Raceme.
   Quantity of flowers and buds per flowering branch.—10 to 15.
Flowers:
   Arrangement.—Flowers form at apical axils, with one flower per axil.
   Flower diameter (horizontal).—1.5 cm.
   Flower height (vertical).—1.0 cm.
   Immature flower.—Upper lobes color (both upper and lower surfaces): More white than RHS NN155D Lower lobes color: Upper surface: More white than RHS NN155D Lower surface: More white than RHS NN155D and very slightly bluish.
   Mature flower.—Upper lobes: Color (both upper and lower surfaces): More white than RHS NN155D Length: 0.9 cm Width: 0.6 cm Shape: Obovate Apex: Obtuse Margin: Entire Texture (both upper and lower surfaces): Smooth Lower lobes: Color: Upper surface: More white than RHS NN155D Lower surface: More white than RHS NN155D and very slightly bluish Length: 0.6 cm Width: 0.2 cm Shape: Obovate Apex: Obtuse Margin: Entire Texture (both upper and lower surfaces): Smooth.
   Corolla.—Color (both inside and outside surfaces): RHS 96D Tube length: 1.0 cm Diameter: 0.25 cm.
   Calyx.—Quantity of sepals: 5 Color (both upper and lower surfaces): RHS 138A Length: 0.9 cm Fused part: 0.4 cm Non-fused part: 0.5 cm Diameter (at fusion point): 0.1 cm Shape: Funnel, star-shaped with fused sepals in lower half Apex: Acute Texture (both upper and lower surfaces): Smooth.
Reproductive organs:
   Androecium.—Quantity of anthers: 5 Filament length: 0.9 cm Filament color: Bluish white Pollen color: Grey Pollen amount: Sparse.
   Gynoecium.—Pistil length: Approximately 0.5 cm Stigma color: Grey Style length: 0.7 cm Style color: RHS 142D.
Fruit and seed set: None observed.
Disease and insect resistance: Typical of species

### COMPARISON WITH PARENTAL VARIETY

‘KLELE12473’ differs from the female parent, ‘LE 05 0429’ (unpatented) in that ‘KLELE12473’ has earlier flowering, very good branching, and weaker stems, whereas ‘LE 05 0429’ has later flowering, moderate branching, and stronger stems.

We claim:

1. A new and distinct lobelia plant named ‘KLELE12473’ as shown and described herein.
